(19) United States
(12) Patent Application Publication
(10) Pub. No.: US 2017/0246192 A1
(43) Pub. Date: Aug. 31, 2017

(54) USE OF ANDROGEN FOR TREATING GASTROINTESTINAL DISORDERS

(71) Applicant: Edward M. Lichten, Birmingham, MI (US)

(72) Inventor: Edward M. Lichten, Birmingham, MI (US)

(21) Appl. No.: 15/456,816

(22) Filed: Mar. 13, 2017

Related U.S. Application Data
(63) Continuation-in-part of application No. 14/035,237, filed on Sep. 24, 2013, now abandoned.
(60) Provisional application No. 61/704,797, filed on Sep. 24, 2012.

(51) Int. Cl. A61K 31/58; A61K 9/00; A61K 31/568

(52) U.S. Cl. CPC A61K 31/58; A61K 31/568; A61K 9/0053; A61K 9/0019

(57) ABSTRACT

By lowering the amount of Sexual Hormone Binding Globulin (SHBG) and, thereafter, raising the serum testosterone level in a person's body, an effective method for treating gastrointestinal disorders such as irritable bowel syndrome and inflammatory bowel disease, including Crohn's Disease and ulcerative colitis, is achieved. First and second androgens, namely, anabolic steroids, are used to lower the SHBG and, thereafter, raise the serum testosterone level in the body, respectively. The SHBG is lowered and the testosterone level is increased until an equilibrium between the SHBG and the second androgen is reached which normalizes the Free Androgen Index (FAI) in the person's body. Preferably, the first androgen used herein to lower the SHBG is stanozolol and the second androgen used herein to normalize the FAI is testosterone.

### CROSS-REFERENCE TO RELATED APPLICATION

This application is a continuation-in-part application which claims the priority benefit of co-pending U.S. Non-Provisional patent application Ser. No. 14/035,237, filed Sep. 24, 2013, for “Use of Androgen For Disease Treatment,” which claims the priority benefit of U.S. Provisional Patent Application Ser. No. 61/704,797, filed Sep. 24, 2012, the entire disclosures of which are hereby incorporated by reference in their entirety.

### BACKGROUND OF THE INVENTION

1. Field of the Invention

The present invention pertains to a method for treating gastrointestinal disorders including irritable bowel syndrome and inflammatory bowel disease. More particularly, the present invention pertains to a method for overcoming exposure to environmental toxins causing gastrointestinal disorders. Even more particularly, the present invention pertains to the use of androgens for lowering the level of Sexual Hormone Binding Globulin (SHBG) in a patient to achieve a suitable Free Androgen Index (FAI) for treating gastrointestinal disorders.

2. Description of Related Art

As is known to those of ordinary skill in the art to which the present invention pertains, there has been reported the use of an androgen, such as stanozolol, for blocking liver production of SHBG. Further, the prior art is replete with reports showing the use of the laboratory measurement of SHBG using an androgen as a marker, sensitivity test, androgen receptor, mutation detection, and so forth. Furthermore, U.S. Pat. Nos. 7,186,706, 6,583,129, and 6,139,873 illustrate the utilization of stanozolol and other androgens in connection with SHBG testing.

Heretofore, toxins, including organic, genetic, environmental, and so forth, have been the subject of many hypotheses in studies determining the cause of gastrointestinal disorders within the gastrointestinal (GI) tract.

The GI tract includes all structures between the mouth and the anus, which forms a continuous passageway including the main organs of digestion, namely, the stomach, small intestine, and large intestine.

Common infections and diseases resulting from overexposure of these environmental toxins include irritable bowel syndrome (IBS) and inflammatory bowel disease (IBD).

IBS is a functional gastrointestinal disorder and includes symptoms such as abdominal pain and changes in the pattern of bowel movements without any evidence of underlying damage. IBS has been classified into four different categories based upon whether diarrhea is common (IBS-D), constipation is common (IBS-C), both are common (IBS-M), or neither occurs very often (IBS-U).

IBS is commonly treated by altering one's diet to avoid certain carbohydrates or consume more fiber, taking medication such as laxatives or those increasing the amount of serotonin in the intestines, or taking probiotics. Oftentimes, IBS can be cured, or at least symptoms controlled, using any one or combination of the above treatment methods.

IBD is an autoimmune disease where the body' s own immune system attacks elements of the digestive system. IBD primarily includes Crohn' s disease and ulcerative colitis. Both Crohn's disease and ulcerative colitis are oftentimes difficult to distinguish from one another as they both present similar symptoms, for example, abdominal pain, vomiting, diarrhea, rectal bleeding, severe internal cramps/muscle spasms in the pelvis region, and weight loss. Nonetheless, the main difference between the two diseases is the location of the inflammatory changes. Crohn's may affect any part of the GI tract while ulcerative colitis is restricted to the colon and the rectum.

Treatment of ulcerative colitis may include surgery where the rectum and colon is removed. This results in the patient having to use an ileostomy bag to collect feces. Crohn's disease, on the other hand, cannot be cured by surgery. Instead, surgery is used to remove the inflamed parts of the intestines to treat complications such as abscesses, strictures, or fisulae.

As is disclosed hereinafter, the predicate for the present invention resides in the postulate that environmental toxins cause gastrointestinal disorders and these toxins affect the body with respect to the levels of SHBG. Therefore, by blocking or lowering the level of SHBG and subsequently raising the testosterone level, the FAI equilibrium in the body is changed, thereby reducing the effects of the environmental toxins and minimizing the effects of these gastrointestinal disorders.

In essence, by utilizing an androgen to lower the SHBG in the body, there is a decrease in the effect of the endocrine disruption compounds, thereby reducing the propensity of the body to become diseased.

More particularly, by reducing the effects of these environmental toxins with regards to lowering the SHBG in the body, a gastrointestinal disorder may be treated and side effects minimized.

It is to this to which the present invention is directed.

### SUMMARY OF THE INVENTION

In accordance with the present invention, there is provided a method for treating gastrointestinal disorders, specifically, IBS and IBD, including Crohn' s disease and ulcerative colitis.

The present method comprises the steps of lowering the SHBG in the body through the utilization of an androgen and, thereafter, raising the serum testosterone level in the body through the use of a second androgen to raise the FAI to an acceptable level.

More specifically, by utilizing selected amounts of the first and the second androgens, it is possible to first lower the amount of SHBG in the body with the first androgen and, once the reduced level is achieved, raising the testosterone level in the body with the second androgen to achieve a specified FAI and an equilibrium between the SHBG and the second androgen.

Preferably, the androgens used herein are steroids. Both naturally occurring and synthetic steroids may be used.

For a more complete understanding of the present invention, reference is made to the following detailed description of the invention.

### DETAILED DESCRIPTION OF THE INVENTION

As noted above, the present invention provides a method for treating gastrointestinal disorders such as IBS and IBD, including Crohn's disease and ulcerative colitis.

To avoid having to resort to surgery or alternative medications commonly employed to treat IBS and IBD, the present method comprises the steps of, first, lowering the SHBG in the body through the utilization of a first androgen and, thereafter, once the SHBG reaches the desired reduced level, utilizing a second androgen to raise the serum testosterone level, thereby reaching an acceptable FAI to manage symptoms of gastrointestinal disorders.

As is known to one of ordinary skill in the art, the FAI in a person is determined by a ratio of the serum testosterone level with respect to the SHBG in that person. In other words, FAI=testosterone level/SHBG. Thus, the severity of gastrointestinal disorders exhibited by IBD and IBS is an inverse relationship between the amount of SHBG in comparison to the serum testosterone level in a patient.

Therefore, by first lowering the amount of SHBG with the first androgen and, then, adding the second androgen into the body to raise the serum testosterone level, a desired FAI may be reached and the effective treatment of gastrointestinal disorders caused by environmental toxins is substantially increased.

More specifically, by utilizing selected amounts of the first androgen, it is possible to lower the amount of SHBG in the body to a predetermined amount.

Generally, the first androgen is administered over a period of time of about four to about eight weeks in order to lower the SHBG to within a range of about 30 nanomoles per liter (nmol/l) to about 45 nmol/l for a female and to within a range of about 5 nmol/l to about 15 nmol/l for a male.

After the treatment with the first androgen to lower the SHBG to the specified range, it is necessary to, then, add the second androgen into the system to raise the serum testosterone level to a normal level.

In practicing the present invention, both natural and synthetic androgens may be used.

Representative natural androgens, for example, dihydroepiandrosterone, androstenedione, androstenediol, androsterone, dihydrotestosterone, testosterone, nandrolone, and the like, may be used herein.

Synthetic androgens that lower the SHBG, such as stanozolol, oxandrin, furzabolin, dianabol, danazol, anadrol, and the like, may also be used. It is possible, although not preferred, to use a combination of both natural and synthetic androgens to lower the SHBG.

In the practice hereof, the first androgen, used to lower the SHBG, is an anabolic steroid. Anabolic steroids are well-known and commercially available.

Among the preferable and useful anabolic steroids for lowering the SHBG in accordance herewith are stanozolol, dianabol, danazol, and anadrol.

Most preferably, stanozolol is used as the first androgen for lowering the SHBG. The stanozolol can be administered either orally or as an injectable.

The gender of the patient or person determines the amount of steroid necessary to lower the SHBG in order to achieve the desired FAI. For a male, the FAI should range from about 1 to about 2. For a female, the FAI should range from about 0.03 to about 0.10. Thus, a male needs to have an SHBG level of from about 5 nmol/l to about 15 nmol/l and a female from about 30 nmol/l to about 45 nmol/l in order to ultimately achieve the desired FAI.

Regardless of gender, generally, from about 4.0 milligrams (mgs) to about 8.0 mgs of the stanozolol is administered orally, daily, for about four to about eight weeks in order to effectuate specific lowering of the SHBG.

Where injections are used, generally, from about 10.0 mgs to about 50.0 mgs are injected, once a week, for about four to about eight weeks.

As noted above, after the SHBG has been reduced to an acceptable level, the second androgen, such as a steroid, is, then, administered into the body to rebalance the equilibrium in the body between the SHBG and the second androgen. Among the useful second androgens are testosterone and nandrolone. Preferably, the second androgen is testosterone.

Depending on the gender of the patient, the amount of testosterone added back into the body will vary.

Where the patient is a male, generally from about 100 mgs to about 250 mgs of “added back” testosterone is administered as an injection, once a week, for about four to about eight weeks.

With a female patient, the amount of testosterone administered will generally range from about 15 mgs to about 70 mgs, once a week, for about four to about eight weeks.

The administration of the first and second androgens may be continued indefinitely until desired levels of SHBG and testosterone have been achieved. Preferably, periodic measurements of SHBG and testosterone levels in the patient are taken in order to determine if the desired ranges have been achieved. It is to be understood that it is well known to those of ordinary skill in the art to measure SHBG and testosterone levels in a patient using any standard blood or saliva test.

By following the steps described herein, generally, in about four to eight weeks, equilibrium between the SHBG and the testosterone is achieved and the FAI will fall within the desired range identified above. Furthermore, by administering the specified amounts of androgen, described above, there is a concomitant “re-balancing” of an appropriate testosterone level in relation to the decrease in SHBG.

It is contemplated that by employing the above method to reduce high levels of SHBG in a person's body and, thereafter, raise the serum testosterone level, a satisfactory FAI can be achieved, thereby treating IBS and IBD. As a result thereof, symptoms and side effects of gastrointestinal disorders may be managed and minimized.

Having thus described the invention, what is claimed is:

1. A method for treating gastrointestinal disorders, comprising the steps of:
(a) administering to a person a predetermined amount of a first androgen over a period of time of from about four to about eight weeks to lower the Sexual Hormone Binding Globulin (SHBG) of the person to a pre-selected level;
(b) thereafter, administering a second androgen to the person over a period of time of from about four to about eight weeks to raise the serum testosterone level of the person to achieve an acceptable Free Androgen Index (FAI) according to the gender of the person; and
wherein achieving an acceptable FAI minimizes the symptoms of a gastrointestinal disorder.

2. The method of claim 1 wherein the gastrointestinal disorder is irritable bowel syndrome.

3. The method of claim 1 wherein the gastrointestinal disorder is inflammatory bowel disease.

4. The method of claim 3 wherein the gastrointestinal disorder is Crohn's disease.

5. The method of claim 3 wherein the gastrointestinal disorder is ulcerative colitis.

6. The method of claim 3 wherein the first and the second androgens are steroids.

7. The method of claim 6 wherein the first androgen is selected from the group consisting of dihydroepiandrosterone, androstenedione, androstenediol, androsterone, dihydrotestosterone, testosterone, nandrolone, stanozolol, oxandrin, furazabol, dioxadol, danazol, and anadrol.

8. The method of claim 6 wherein the second androgen is selected from the group consisting of testosterone and nandrolone.

9. The method of claim 8 wherein the first androgen is stanozolol.

10. The method of claim 8 wherein the second androgen is testosterone.

11. The method of claim 6 wherein the first androgen is administered orally in an amount ranging from about 4.0 milligrams to about 8.0 milligrams daily for about four to eight weeks to lower the SHBG.

12. The method of claim 11 wherein the person is male and the second androgen is administered as an injection in an amount ranging from about 100 milligrams to about 250 milligrams, once a week, for about four to about eight weeks.

13. The method of claim 11 wherein the person is a female and the second androgen is administered as an injection in an amount which ranges from about 15 milligrams to about 70 milligrams, once a week, for about four to about eight weeks.

14. The method of claim 3 wherein the SHBG is lowered to about 5 nanomoles per liter to about 15 nanomoles per liter for a male and to about 30 nanomoles per liter to about 45 nanomoles per liter for a female.

15. The method of claim 3 wherein the FAI after treatment ranges from about 1 to about 2 for a male and from about 0.03 to about 0.10 for a female.
